(19) United States
(12) Patent Application Publication
(10) Pub. No.: US 2011/0009788 A1
(43) Pub. Date: Jan. 13, 2011

(54) Orthotic Device with Removably Attachable Actuator

(75) Inventors: Stephen R. Kelly, Manchester, MA (US); Gene K. Tacy, Weare, NH (US); Jamie G. Nichol, Arlington, MA (US)

Correspondence Address: Sunstein Kann Murphy & Timbers LLP, 125 SUMMER STREET, BOSTON, MA, 02110-1618, US

(73) Assignee: MYOMO, INC., Boston, MA (US)

(21) Appl. No.: 12/834,263

(22) Filed: Jul. 12, 2010

Related U.S. Application Data
(60) Provisional application No. 61/224,963, filed on Jul. 13, 2009.

(51) Int. Cl. A61F 5/00

(52) U.S. Cl. 602/16

(57) ABSTRACT

An orthotic device adapted to use one or more actuators includes a frame having a first section and a second section movably coupled to the first section in a coupling region to permit relative motion of the sections about a pivot axis. The frame is configured to receive at least one removably attachable actuator. The orthotic device also includes an actuator removably attachable to the frame. The removably attachable actuator is configured to move the sections relative to one another via a coupling in the coupling region. The removably attachable actuator may be a motorized actuator, a dynamic actuator with at least one spring, or both.

### CROSS REFERENCE TO RELATED APPLICATIONS

The present application claims priority to U.S. Provisional Patent Application No. 61/224,963 filed Jul. 13, 2009, the disclosure of which is incorporated by reference herein in its entirety.

### TECHNICAL FIELD

The present invention generally relates to orthotic devices and, more particularly, the invention relates to orthotic devices with a removably attachable actuator.

### BACKGROUND ART

Stroke, brain injury, and other neuromuscular trauma survivors are often left with hemipareisis, or severe weakness in certain parts of the body. The result can be impaired or lost function in one or more limbs. It has been shown that people can rehabilitate significantly from many of the impairments following such neurological traumas. Further, it has been shown that rehabilitation is much more effective, and motor patterns re-learned more quickly, if the rehabilitative exercise regime includes the execution of familiar and functional tasks. Following neuromuscular trauma, however, the control or strength in the afflicted limb or limbs may be so severely diminished that the patient may have difficulty performing (or be completely unable to perform) constructive, functional rehabilitation exercises without assistance.

Over the course of rehabilitation, a patient may benefit from various exercises that may be performed with different kinds of orthotic devices. Having a patient borrow or purchase various orthotic devices, however, may become unduly burdensome and cost prohibitive.

### SUMMARY OF EMBODIMENTS

In accordance with one embodiment of the invention, an orthotic device adapted to use one or more actuators includes a frame having a first section and a second section movably coupled to the first section in a coupling region to permit relative motion of the sections about a pivot axis. The frame is configured to receive at least one removably attachable actuator. The orthotic device also includes an actuator removably attachable to the frame. The removably attachable actuator is configured to move the first section and the second section relative to one another via a coupling in the coupling region.

In related embodiments, the removably attachable actuator may be a motorized actuator. The motorized actuator may include a rechargeable battery that provides energy to the motorized actuator. Alternatively, or in addition, the removably attachable actuator may be at least one dynamic actuator with at least one spring. The dynamic actuator may further include an adjustment mechanism that changes a force profile of the at least one spring. The orthotic device may further include at least one electromyographic sensor in communication with the removably attachable actuator. The electromyographic sensor may be configured to sense an electromyographic signal from one or more muscles of a user, and the removably attachable actuator may be configured to move the sections relative to one another based on the sensed signal. The orthotic device may further include an attachment mechanism coupled to the frame. The attachment mechanism may be configured to hold the frame to a body part of a user. The at least one electromyographic sensor may be coupled to the attachment mechanism. The attachment mechanism may be a cuff, at least one strap, or both.

In accordance with another embodiment of the invention, an orthotic device adapted to use at least two different types of actuators includes a frame having a first section and a second section movably coupled to the first section in a coupling region to permit relative motion of the sections about a pivot axis. The frame is configured to receive at least one removably attachable actuator. The orthotic device further includes an actuator removably attachable to the frame. The removably attachable actuator is configured to move the first section and the second section relative to one another via a coupling in the coupling region. The removably attachable actuator is a motorized actuator having a drive assembly and a motor coupled to the drive assembly. One portion of the drive assembly is coupled to the frame, and the motor is positioned beneath the frame and substantially parallel to the pivot axis of the frame. In related embodiments, the orthotic device may further include a second actuator removably attachable to the frame. The second removably attachable actuator may be a dynamic actuator with at least one spring.

In accordance with another embodiment of the invention, an orthotic device adapted to use at least two different types of actuators includes a frame having a first section and a second section movably coupled to the first section in a coupling region to permit relative motion of the sections about a pivot axis. The frame is configured to receive at least one removably attachable actuator. The orthotic device further includes an actuator removably attachable to the frame. The removably attachable actuator is configured to move the first section and the second section relative to one another via a coupling in the coupling region. The removably attachable actuator is at least one dynamic actuator with at least one spring. In related embodiments, the orthotic device may further include a second actuator removably attachable to the frame. The second removably attachable actuator may be a motorized actuator having a drive assembly and a motor coupled to the drive assembly. One portion of the drive assembly is coupled to the frame, and the motor is positioned beneath the frame and substantially parallel to the pivot axis of the frame.

### BRIEF DESCRIPTION OF THE DRAWINGS

The foregoing features of the invention will be more readily understood by reference to the following detailed description, taken with reference to the accompanying drawings, in which:

FIG. 1 schematically shows a perspective view of an orthotic device frame according to embodiments of the present invention;

FIG. 2 schematically shows a side view of an orthotic device frame in one position according to embodiments of the present invention;

FIG. 3 schematically shows a side view of an orthotic device frame in another position according to embodiments of the present invention;

FIG. 4 schematically shows a perspective view of an orthotic device frame with straps according to embodiments of the present invention;

FIG. 5 schematically shows a perspective view of an orthotic device frame with straps and a cuff according to embodiments of the present invention;

FIG. 6 schematically shows a perspective view of an orthotic device frame with an attached motorized actuator according to embodiments of the present invention;

FIG. 7 schematically shows a perspective side view of an orthotic device frame with an attached motorized actuator according to embodiments of the present invention;

FIG. 8 shows the orthotic device of FIG. 7 with the housing removed from the motorized actuator;

FIG. 9 schematically shows a perspective view of the motorized actuator of FIG. 8 according to embodiments of the present invention;

FIG. 10 schematically shows a bottom perspective view of FIG. 9;

FIG. 11 schematically shows a perspective view of an orthotic device frame with two attached dynamic actuators according to embodiments of the present invention;

FIG. 12 schematically shows a perspective view of the orthotic device of FIG. 11 with the housings removed from the dynamic actuators;

FIG. 13 schematically shows a side view of the orthotic device of FIG. 12 according to embodiments of the present invention;

FIG. 14 schematically shows a perspective top view of the dynamic actuator of FIG. 13 according to embodiments of the present invention;

FIG. 15 schematically shows a perspective bottom view of FIG. 14;

FIG. 16 schematically shows a perspective top view of a dynamic member according to embodiments of the present invention;

FIG. 17 schematically shows a perspective side view of FIG. 16; and

FIG. 18 is a graph showing various force vs. position profiles for the orthotic device when using one or more dynamic actuators according to embodiments of the present invention.

### DESCRIPTION OF ILLUSTRATIVE EMBODIMENTS

Various embodiments of the present invention provide an orthotic device having a removably attachable actuator. This allows the configuration of the device to be easily changed between one type of actuator and another, for example, a motorized actuator and a dynamic actuator. The orthotic device includes a frame having two sections movably coupled to each other and an actuator removably attachable to the frame. The removably attachable actuator is configured to move the sections relative to one another. The frame is configured to receive one or more actuators where the two sections are movably coupled together. Each section of the frame is intended to be attached to a body part of a user such that the orthotic device moves the two parts relative to one another about a joint, e.g., upper and lower arm about the elbow, upper and lower leg about the knee, foot and lower leg about the ankle. Details of illustrative embodiments are discussed below.

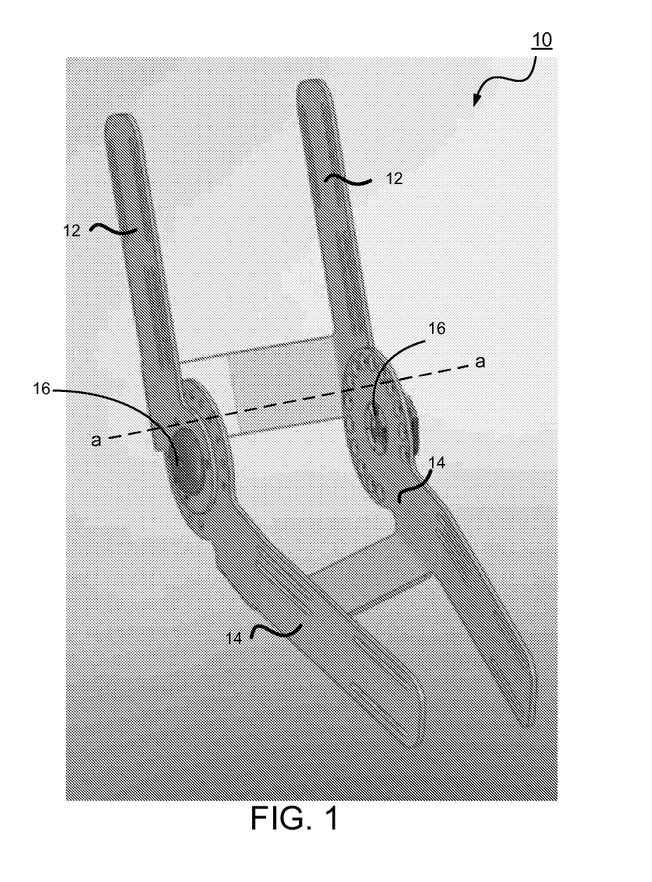

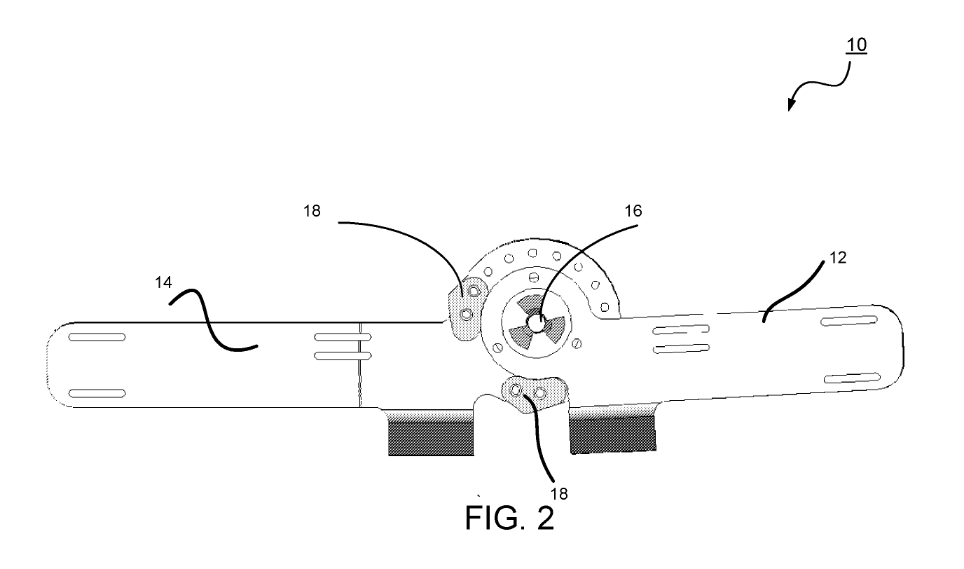

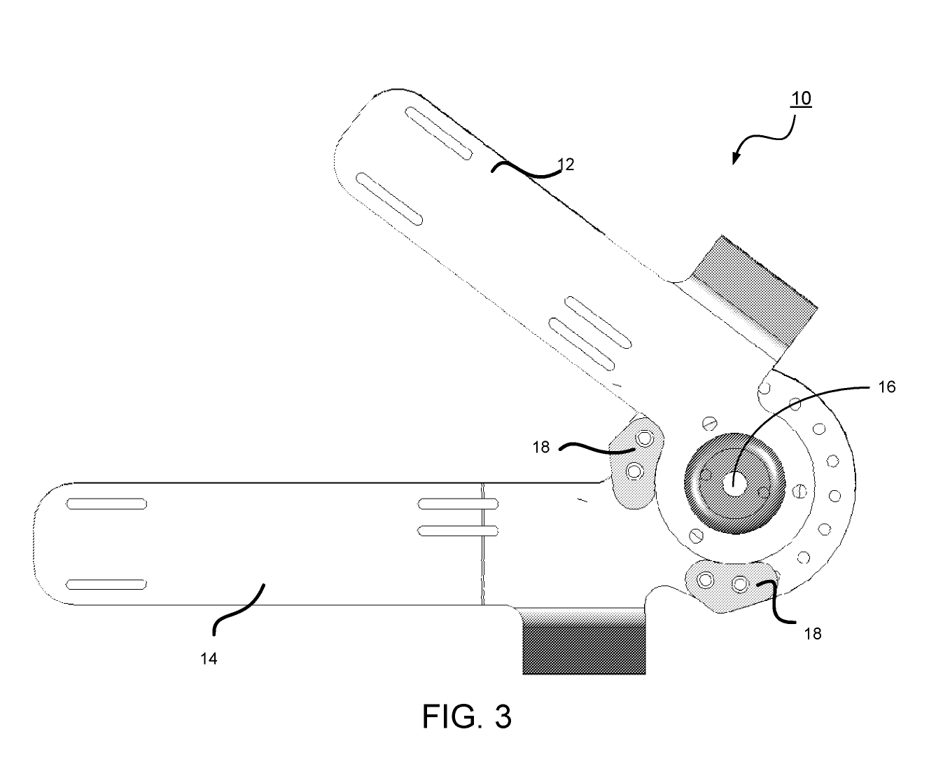

FIG. 1 schematically shows a perspective view of an orthotic device frame 10 according to embodiments of the present invention. The frame 10 has a upper section 12 and a lower section 14 movably coupled to the upper section 12 at two coupling regions 16. The upper section 12 and the lower section 14 move with respect to one another about a pivot axis, designated as dotted line a-a. The frame 10 is intended to be secured to the user so that the body parts are placed between the two parts of sections 12 and 14 and the joint is placed near the pivot axis and between the two coupling regions 16. As shown in FIGS. 2 and 3, the frame 10 may have physical stops 18 that limit the range of motion of the sections 12, 14 with respect to one another. For example, FIG. 2 shows the frame 10 in one position where the sections 12, 14 are approximately 180 degrees apart, and FIG. 3 shows the frame 10 in another position where the sections are approximately 45 degrees apart, although the stops 18 may be placed at other positions allowing other ranges of motion. This allows the orthotic device to limit the motion of the two body parts, and allows that motion to be varied during the course of rehabilitation or set depending on the condition of the user.

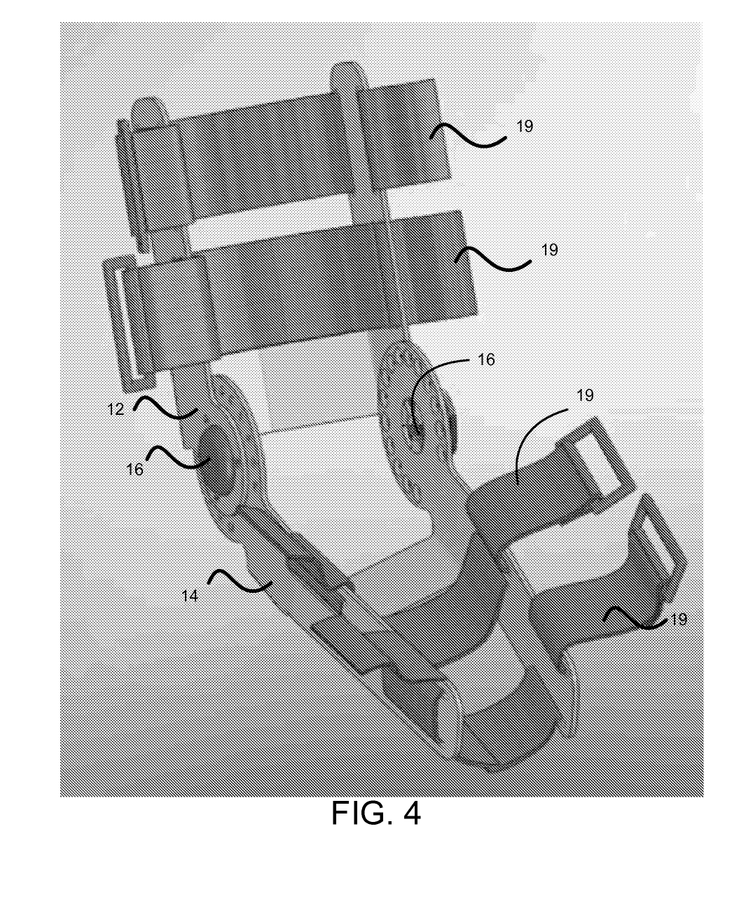

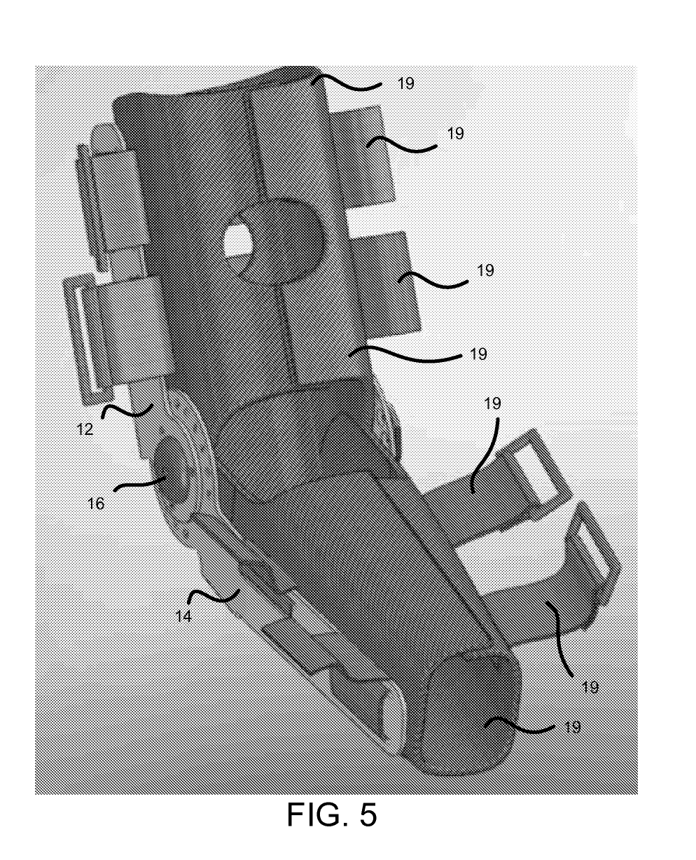

The frame 10 may be secured to the user by an attachment mechanism 19 that is coupled to the upper section 12 and the lower section 14. For example, as shown in FIG. 4, the attachment mechanism 19 may be one or more straps that secure each of the sections 12, 14 to one body part of the user. The attachment mechanism 19 may be a cuff, such as shown in FIG. 5, that may be used in addition to, or instead of, the straps.

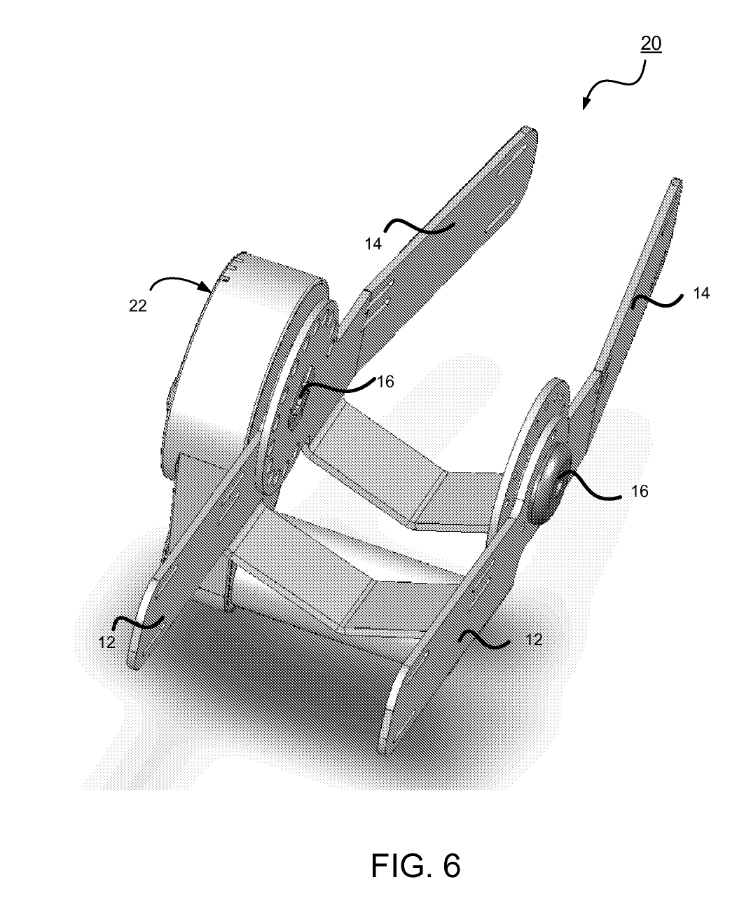

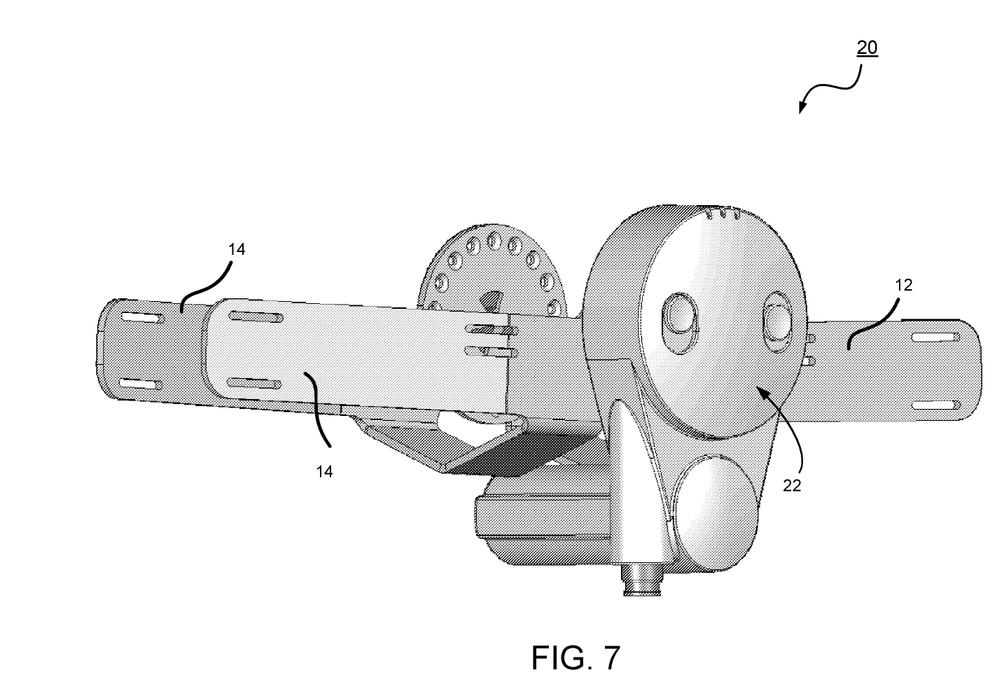

FIGS. 6 and 7 schematically show a perspective top view and a perspective side view, respectively, of an orthotic device 20 according to embodiments of the present invention. For clarity, the remaining figures show the frame 10 without the attachment mechanism 19. As shown, a motorized actuator 22 is coupled to the frame 10 at one of the coupling regions 16 where the sections 12, 14 are movably coupled together. The motorized actuator 22 is configured to be removably attached to the frame 10 so that the device 20 may be easily changed from one type or configuration of actuator to another. The motorized actuator 22 is coupled to the frame 10 on the outside of the frame 10, away from where the user places their body part on the inside of the frame 10. Although the motorized actuator 22 is shown on one side of the frame 10 in FIGS. 6 and 7, an actuator may be used on one side or the other of the frame 10, or both sides of the frame 10, depending on the desired configuration of the orthotic device 20.

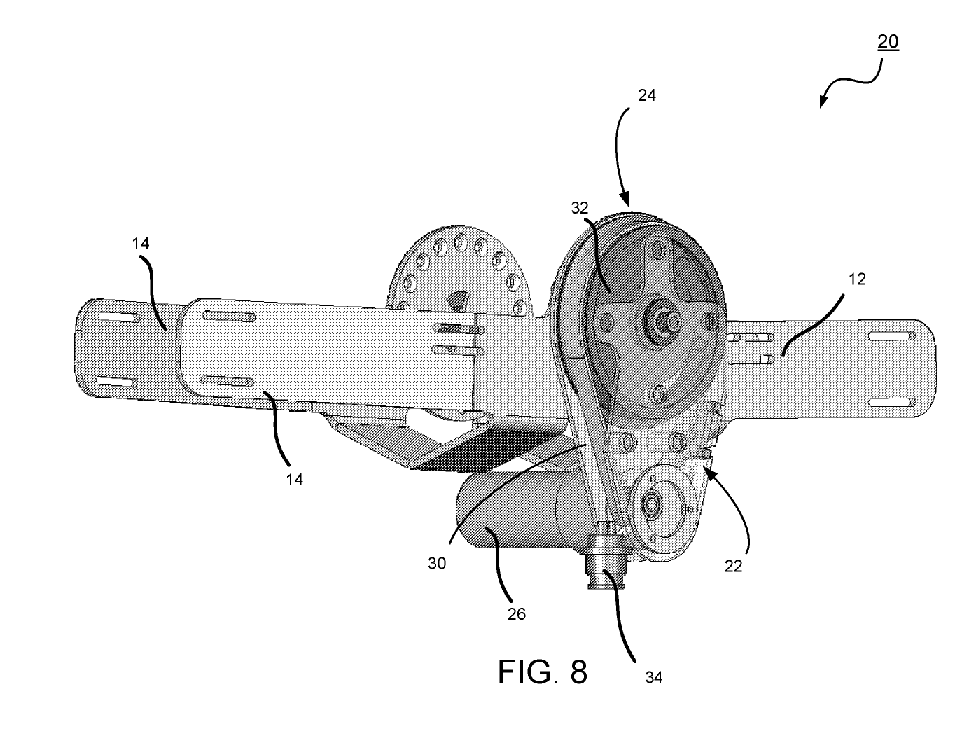

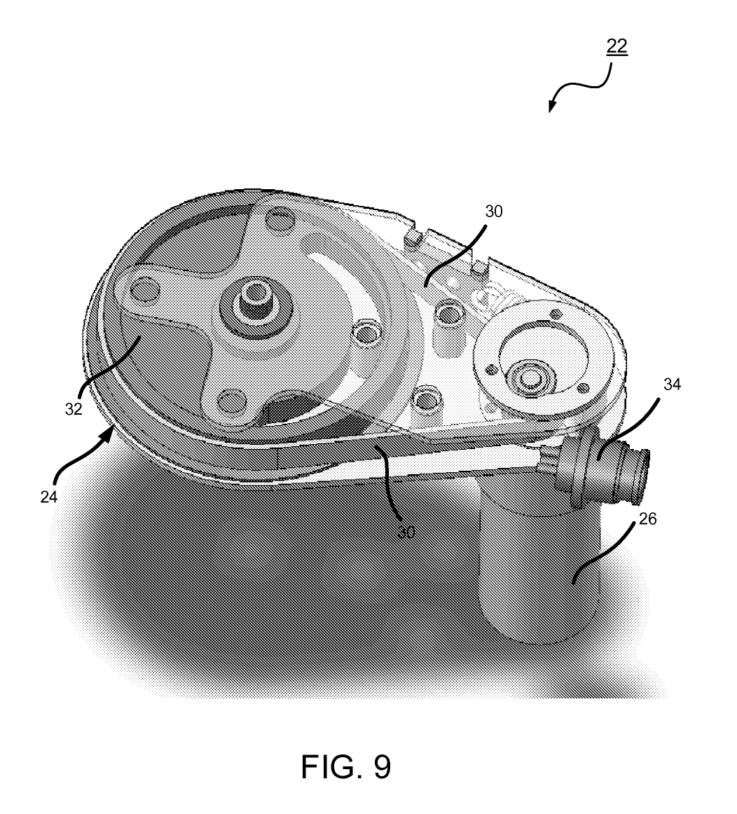

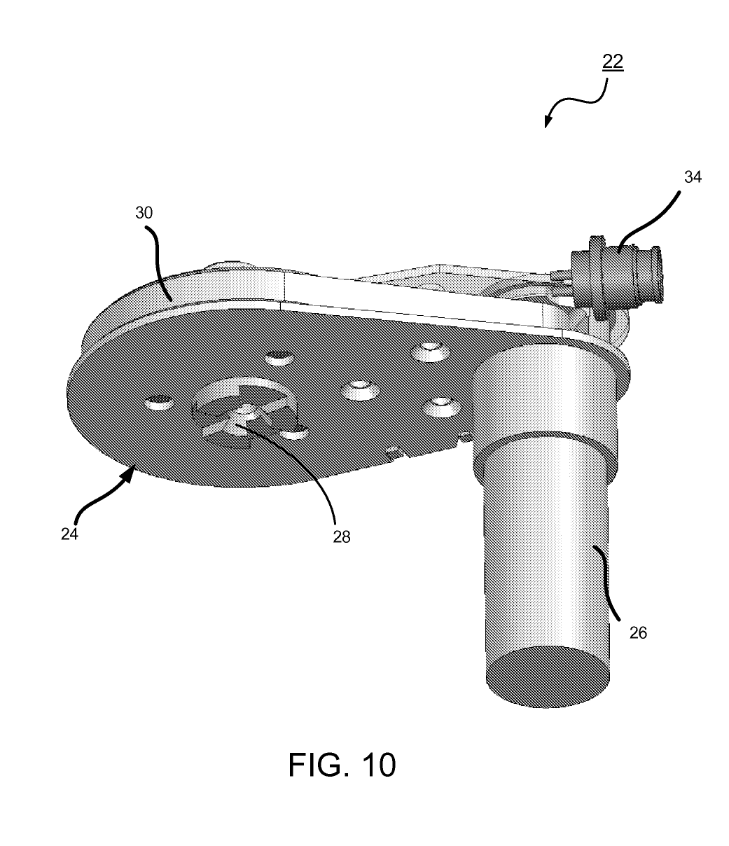

FIG. 8 shows the orthotic device of FIG. 7 with the housing removed from the motorized actuator 22. As shown, the motorized actuator 22 includes a drive assembly 24 and a motor 26 coupled to the drive assembly 24. The drive assembly 24 is coupled to the frame 10 at a coupling area 28 in the coupling region 16. As shown in FIGS. 6-8, the motor 26 is positioned beneath the frame 10 such that the motor 26 is substantially parallel to the pivot axis of the frame 10. FIG. 9 schematically shows a perspective view of the motorized actuator 22 of FIG. 8 not attached to the frame 10, and FIG. 10 schematically shows a bottom perspective view of FIG. 9. As shown, the drive assembly 24 may include a belt 30 and a pulley 32 system, such that the motor 26 engages the belt 30, and the belt 30 causes the pulley 32 to rotate as known by those skilled in the art. The rotating pulley 32 then causes the sections 12, 14 to move relative to one another via the coupling area 28 in the coupling region 16. Although a belt 30 and pulley 32 system are shown, other rotating drive assembly mechanisms may be used. The motorized actuator 22 may also include a power connector 34 that is electrically coupled to the motor 26. The power connector 34 may provide a connection to a power supply (not shown) that provides energy to the motor 26. Alternatively, or in addition, the motorized actuator 22 may include a battery (not shown), preferably a rechargeable battery, electrically coupled to the motor 26 that provides energy to the motor 26. The battery may be recharged via the power connector 34.

The orthotic device 20 may also include at least one electromyographic sensor (not shown) which may be in communication with the motor 26 in the motorized actuator 22. The at least one electromyographic sensor is configured to sense an electromyographic signal from one or more muscles of the user. The sensor may be adjacent to the skin of the user or implanted under the skin of the user. The sensor may be positioned within the attachment mechanism 19 so that the sensor is adjacent to the skin of the user or may be placed at some other location. The motorized actuator 22 may be configured to move the sections 12, 14 relative to one another based on the sensed signal.

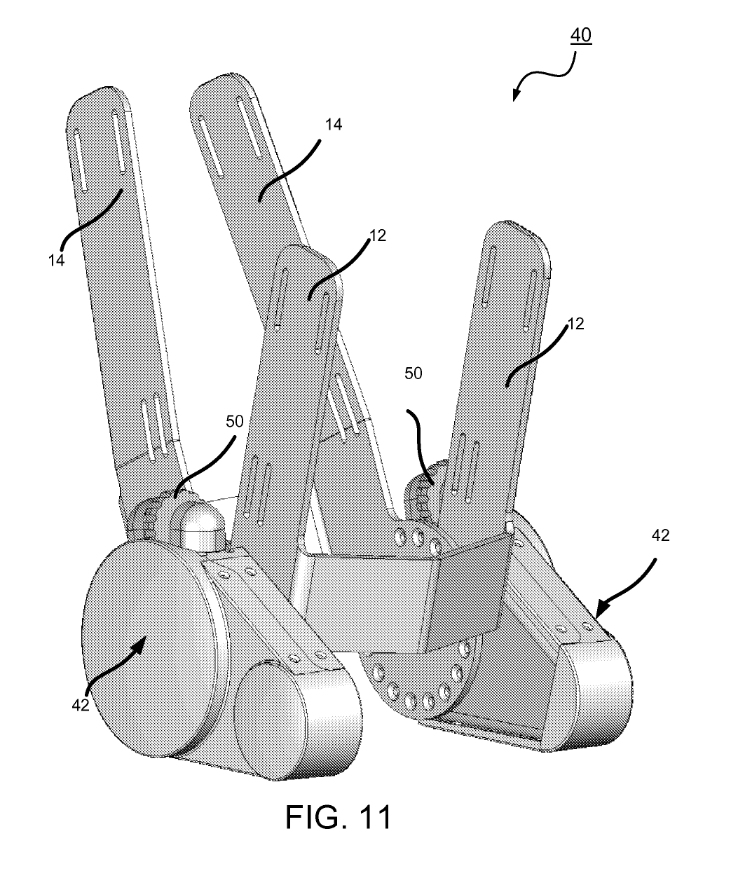

FIG. 11 schematically shows a perspective view of another orthotic device 40 with two dynamic actuators 42 attached to the frame 10 according to embodiments of the present invention. As shown, each dynamic actuator 42 is coupled to the frame 10 at one of the coupling regions 16 where the sections 12, 14 are movably coupled together. Each dynamic actuator 42 is configured to be removably attached to the frame 10 so that the device 40 may be easily changed from one type or configuration of actuator to another. Each dynamic actuator 42 is coupled to the frame 10 on the outside of the frame 10. Although two dynamic actuators 42 are shown on both sides of the frame 10 in FIG. 11, only one dynamic actuator 42 may be used on one side or the other of the frame 10, depending on the desired configuration of the orthotic device 40. Alternatively, one dynamic actuator 42 may be used on one side of the frame 10 and a motorized actuator 22 may be used on the other side of the frame 10. Although a motorized actuator 22 and a dynamic actuator 42 are discussed herein, other types or configurations of actuators may be used instead of, or in addition to, those shown and described.

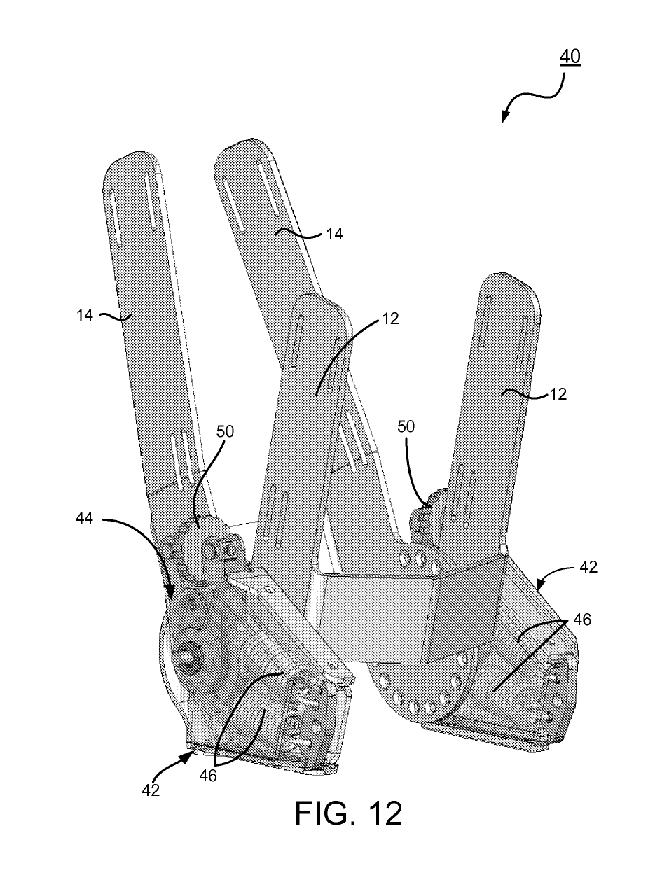

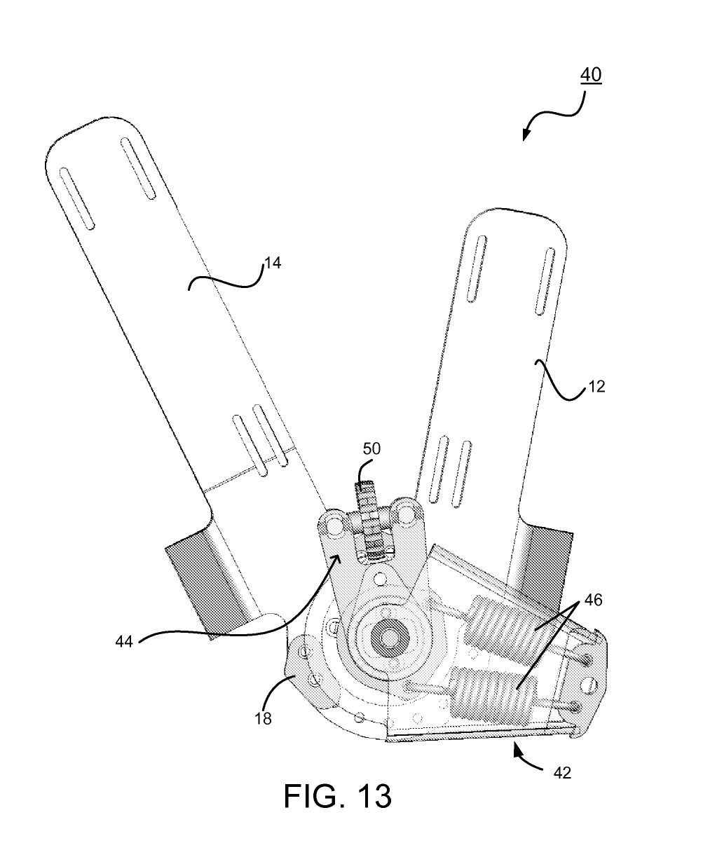

FIGS. 12 and 13 schematically show a perspective view and a side view, respectively, of the orthotic device 40 of FIG. 11 with the housings removed from the dynamic actuators 42. As shown, the dynamic actuator 42 includes a dynamic member 44 coupled to at least one spring 46. The dynamic member 44 allows the direction and the magnitude of the force necessary to move the sections 12, 14 relative to one another to be changed in the dynamic actuator 42. For example, the dynamic actuator(s) 42 may be configured so that the user must exert a force to move the sections 12, 14 apart from one another (e.g., such as shown in FIG. 2), and the at least one spring 46 moves the sections 12, 14 closer together. The dynamic actuator(s) 42 may be configured so that the user must exert a force to move the sections 12, 14 closer to one another (e.g., such as shown in FIG. 3), and the at least one spring 46 moves the sections 12, 14 apart from one another. The dynamic actuator(s) 42 may also be configured so that the user must exert a force to move the sections 12, 14 in either direction. For example, one of the dynamic actuators 42 may be configured so that the user must exert a force to move the sections 12, 14 apart from one another while the other dynamic actuator 42 may be configured so that the user must exert a force to move the sections 12, 14 closer to one another.

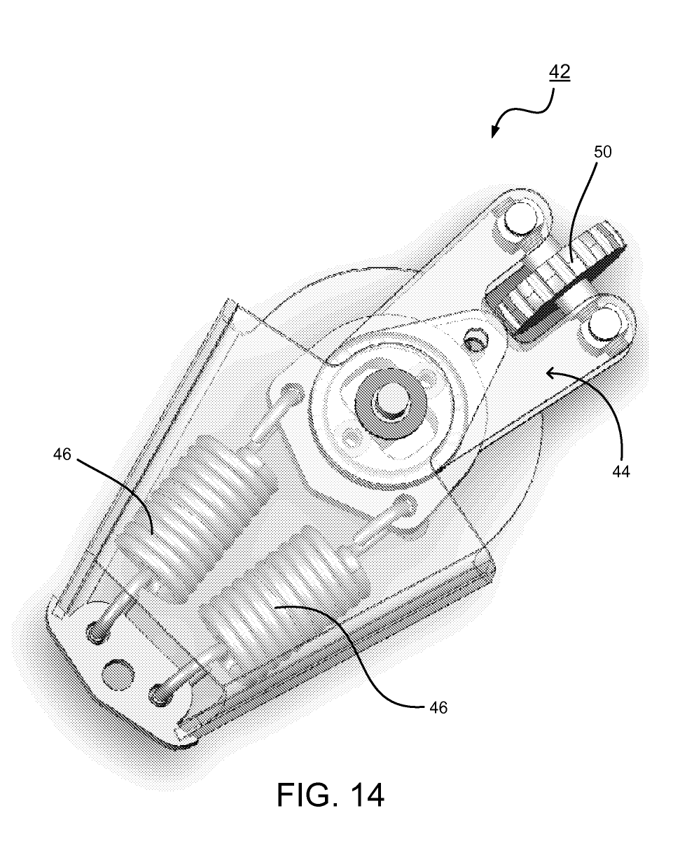

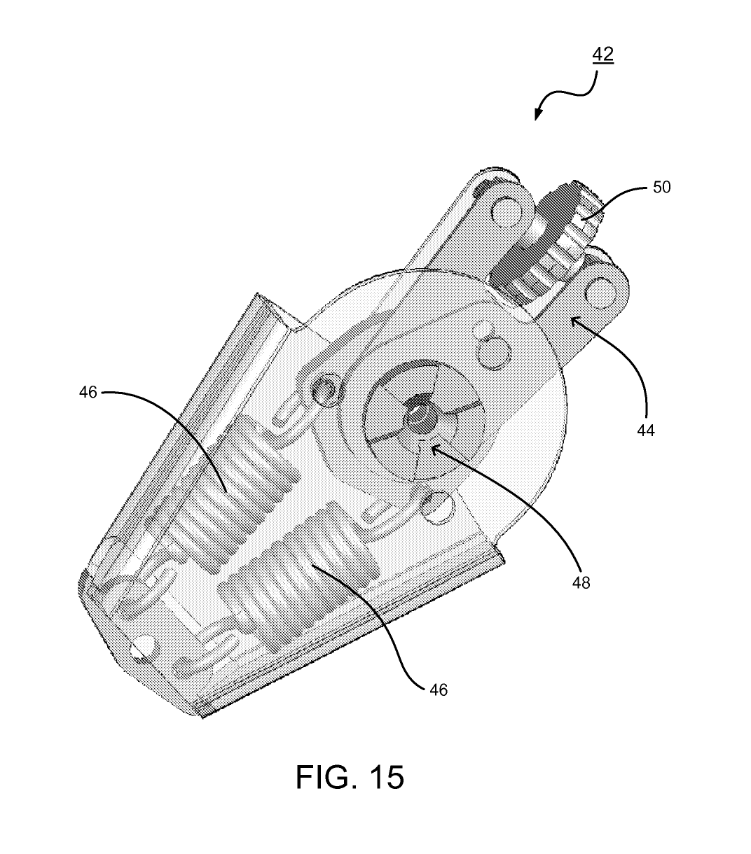

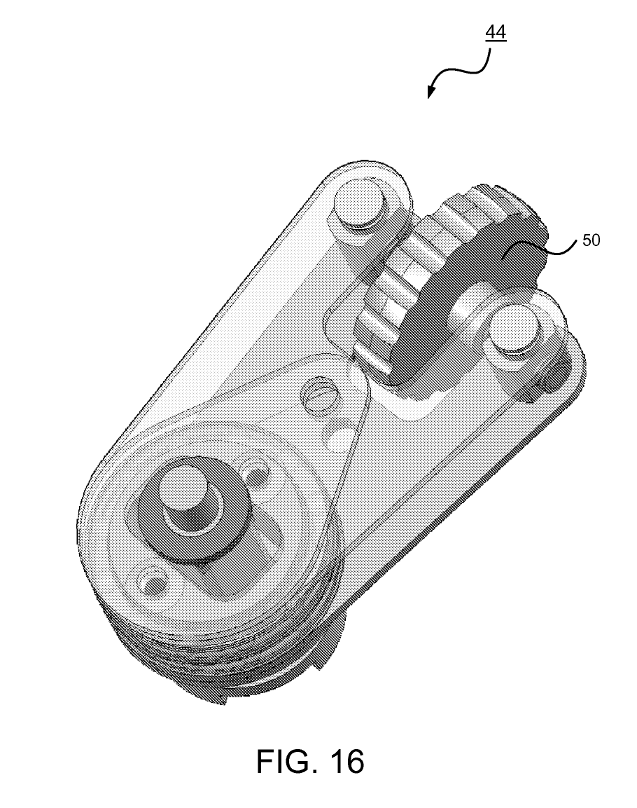

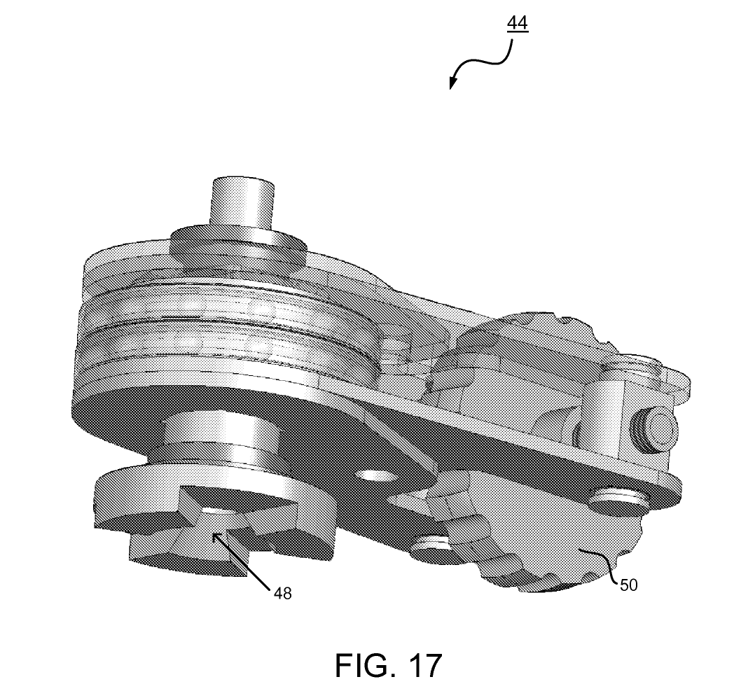

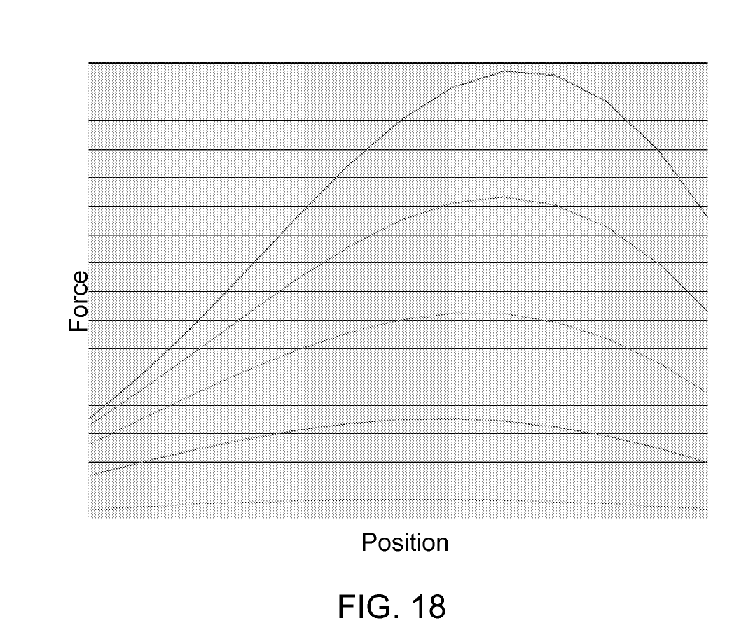

FIG. 14 schematically shows a perspective top view of the dynamic actuator 42 of FIG. 13 not attached to the frame 10, and FIG. 15 schematically shows a perspective bottom view of FIG. 14. Similarly, FIG. 16 schematically shows a perspective top view of a dynamic member 44, and FIG. 17 schematically shows a perspective side view of FIG. 16. As shown in FIGS. 12-17, the dynamic actuator 42 is coupled to the frame 10 at a coupling area 48 in the coupling region 16. The dynamic member 44 also includes an adjustment mechanism 50 that causes the direction and the magnitude of the force necessary to move the sections 12, 14 relative to one another to be changed. For example, when the adjustment mechanism 50 is moved in one direction, at least one of the springs 46 may be lengthened, which may subsequently cause the user to exert more force to further lengthen the springs 46 when attempting to move the sections 12, 14 relative to one another. Similarly, when the adjustment mechanism 50 is moved in the other direction, or further in the same direction, at least one of the springs 46 may be placed in a relaxed position under little to no tension force. Thus, the adjustment mechanism 50 allows a continuously varying change in the tension applied to at least one of the springs 46, which allows both the magnitude and the direction of the force necessary to move the sections 12, 14 relative to one another to be changed. Accordingly, the orthotic device 40 having one or more dynamic actuators 42 may be configured to have several force vs. position profiles, such as shown in FIG. 18, although other force profiles may also be used depending on the type or configuration of the actuator attached to the frame 10.

Although the above discussion discloses various exemplary embodiments of the invention, it should be apparent that those skilled in the art can make various modifications that will achieve some of the advantages of the invention without departing from the true scope of the invention. For example, the sections 12, 14 of the frame 10 are described as coupled together at two coupling regions 16 that move with respect to one another about a pivot axis. However, the sections 12, 14 may be coupled together using other constraint mechanisms, e.g., a universal joint, a sliding (prismatic) joint, etc.

What is claimed is:

1. An orthotic device adapted to use one or more actuators, the orthotic device comprising:
a frame having a first section and a second section movably coupled to the first section in a coupling region to permit relative motion of the sections about a pivot axis, the frame configured to receive at least one removably attachable actuator; and
an actuator removably attachable to the frame in the coupling region, the removably attachable actuator configured to move the sections relative to one another via a coupling in the coupling region.

2. The orthotic device of claim 1, wherein the removably attachable actuator is a motorized actuator.

3. The orthotic device of claim 2, wherein the motorized actuator includes a rechargeable battery that provides energy to the motorized actuator.

4. The orthotic device of claim 1, wherein the removably attachable actuator is at least one dynamic actuator with at least one spring.

5. The orthotic device of claim 4, wherein the at least one dynamic actuator further includes an adjustment mechanism that changes a force profile of the at least one spring.

6. The orthotic device of claim 1, further comprising at least one electromyographic sensor in communication with the removably attachable actuator, the at least one electromyographic sensor configured to sense an electromyographic signal from one or more muscles of a user, the removably attachable actuator configured to move the sections relative to one another based on the sensed signal.

7. The orthotic device of claim 6, further comprising an attachment mechanism coupled to the frame, the attachment mechanism configured to hold the frame to a body part of a user, wherein the at least one electromyographic sensor is coupled to the attachment mechanism.

8. The orthotic device of claim 1, further comprising an attachment mechanism coupled to the frame, the attachment mechanism configured to hold the frame to a body part of a user.

9. The orthotic device of claim 8, wherein the attachment mechanism is a cuff, at least one strap or both.

10. An orthotic device adapted to use at least two different types of actuators, the orthotic device comprising:
a frame having a first section and a second section movably coupled to the first section in a coupling region to permit relative motion of the sections about a pivot axis, the frame configured to receive at least one removably attachable actuator; and
an actuator removably attachable to the frame in the coupling region, the removably attachable actuator configured to move the sections relative to one another via a coupling in the coupling region, wherein the removably attachable actuator is a motorized actuator, the motorized actuator having a drive assembly and a motor coupled to the drive assembly, one portion of the drive assembly coupled to the frame, and the motor positioned beneath the frame and substantially parallel to the pivot axis of the frame.

11. The orthotic device of claim 10, wherein the motorized actuator includes a rechargeable battery that provides energy to the motorized actuator.

12. The orthotic device of claim 10, further comprising at least one electromyographic sensor in communication with the motor in the motorized actuator, the at least one electromyographic sensor configured to sense an electromyographic signal from one or more muscles of a user, the motorized actuator configured to move the sections relative to one another based on the sensed signal.

13. The orthotic device of claim 12, further comprising an attachment mechanism coupled to the frame, the attachment mechanism configured to hold the frame to a body part of a user, wherein the at least one electromyographic sensor is coupled to the attachment mechanism.

14. The orthotic device of claim 10, further comprising a second actuator removably attachable to the frame, wherein the second removably attachable actuator is a dynamic actuator with at least one spring.

15. An orthotic device adapted to use at least two different types of actuators, the orthotic device comprising:
a frame having a first section and a second section movably coupled to the first section in a coupling region to permit relative motion of the sections about a pivot axis, the frame configured to receive at least one removably attachable actuator; and
an actuator removably attachable to the frame in the coupling region, the removably attachable actuator configured to move the sections relative to one another via a coupling in the coupling region, wherein the removably attachable actuator is at least one dynamic actuator with at least one spring.

16. The orthotic device of claim 15, wherein the at least one dynamic actuator further includes an adjustment mechanism that changes a force profile of the at least one spring.

17. The orthotic device of claim 15, further comprising a second actuator removably attachable to the frame, wherein the second removably attachable actuator is a motorized actuator, the motorized actuator having a drive assembly and a motor coupled to the drive assembly, one portion of the drive assembly coupled to the frame, and the motor positioned beneath the frame and substantially parallel to the pivot axis of the frame.

18. The orthotic device of claim 15, further comprising at least one electromyographic sensor in communication with the motor in the motorized actuator, the at least one electromyographic sensor configured to sense an electromyographic signal from one or more muscles of a user, the motorized actuator configured to move the sections relative to one another based on the sensed signal.
